(19) United States
(12) Patent Application Publication
(10) Pub. No.: US 2024/0321405 A1
(43) Pub. Date: Sep. 26, 2024

(54) QUALITY ASSESSMENT OF AROMA MOLECULES

(71) Applicant: BASF SE, Ludwigshafen Am Rhein (DE)

(72) Inventors: Oliver Sala, Ludwigshafen (DE); Florian Garlichs, Lampertheim (DE); Miriam Mathea, Ludwigshafen (DE); Rebecca Sure, Ludwigshafen (DE)

(21) Appl. No.: 18/574,450

(22) PCT Filed: Jun. 23, 2022
(86) PCT No.: PCT/EP2022/067251

§ 371 Date: Dec. 27, 2023

(30) Foreign Application Priority Data
Jun. 28, 2021 (EP) 21181950.3

(51) Int. Cl. G16C 20/30; G01N 33/00; G16C 20/70

(52) U.S. Cl. CPC G16C 20/30; G01N 33/0001; G16C 20/70

(57) ABSTRACT

The present invention relates to a computer-implemented for quality assessment of aroma molecules using single-molecule olfactory predictions comprising (i) receiving input data, preferably via an input unit (10), of at least one set of odorant descriptions (A) obtained by olfactory prediction of an aroma molecule and a second set of odorant descriptions (B) obtained by human olfactory evaluation (ii) comparing the received set of odorant descriptions (A) obtained by olfactory prediction of the aroma molecule to the second set of odorant descriptions (B) obtained by olfactory prediction of the same aroma molecule (iii) determining quality of aroma molecule based on the discrepancy between the at least one set of odorant descriptions (A) obtained by olfactory prediction of the aroma molecule and the second set of odorant descriptions (B) obtained by human olfactory evaluation via a processing unit (20a) (iv) providing, preferably via an output unit (30), quality assessment results of the aroma molecule.

The present invention relates to a computer-implemented method for quality assessment of aroma molecules using single-molecule olfactory predictions comprising (i) receiving input data, preferably via an input unit, of at least one set of odorant descriptions (A) obtained by olfactory prediction of an aroma molecule and a second set of odorant descriptions (B) obtained by human olfactory evaluation; (ii) comparing the received set of odorant descriptions (A) obtained by olfactory prediction of the aroma molecule to the second set of odorant descriptions (B) obtained by olfactory prediction of the same aroma molecule; (iii) determining, specifically calculating, quality of aroma molecule based on the discrepancy between the at least one set of odorant descriptions (A) obtained by olfactory prediction of the aroma molecule and the second set of odorant descriptions (B) obtained by human olfactory evaluation via a processing unit (20), and (iv) providing, preferably via an output unit (30), quality assessment results of the aroma molecule. The present invention also relates to an apparatus for quality assessment of aroma molecules using single-molecule olfactory predictions, comprising:—an input unit configured to receive a data input, preferably a user interface, wherein the data input comprises at least one set of odorant descriptions (A) obtained by olfactory prediction of an aroma molecule and a second set of odorant descriptions (B) obtained by human olfactory evaluation;—a processing unit, preferably a processing unit comprising at least one processor configured, specifically by programming, to compare the received set of odorant descriptions (A) obtained by olfactory prediction of an aroma molecule to the second set of odorant descriptions (B) obtained by olfactory prediction of the same aroma molecule and determine, specifically to calculate, the quality of aroma molecule based on the discrepancy between the at least one set of odorant descriptions (A) obtained by olfactory prediction of the aroma molecule and the second set of odorant descriptions (B) obtained by human olfactory evaluation; and—an output unit configured to output the quality assessment results of the aroma molecule to the user and/or to a data interface; and to a system comprising said apparatus. The present invention further relates to methods, computer programs, data carriers, and uses related to the aforesaid method, apparatus, and system.

Quality control of aroma molecules, as well as the products derived from them, comprises the comparison of sensory, analytical, and, if necessary, microbiological data with standards and specifications. Aroma molecules are identified by generally accepted analytical parameters such as relative density, refractive index, optical rotation, and melting point. Spectroscopic methods such as infrared (IR) and near-infrared (NIR) are becoming more important for fast identification checks. Standardization of specifications for complex aroma molecules, such as essential oils and absolutes, is far more difficult than for single compounds. Gas chromatography-olfacto-metry (GC-O) has become a standard method for the detection and determination of sensory important constituents in complex mixtures. Special detector systems such as electron capture detector (ECD) and atomic absorption spectroscopy (AAS) are used for the detection and quantification of halogen and heavy metal content.

Quality control of aroma molecules is important for perfume manufacturers, as well as for their customers, to assure that the finished perfume is the one that was formulated. On the other hand, analytical methods are necessary to assure, for safety purposes, that there are no undesired or banned compounds present in the finished product.

U.S. Pat. No. 6,558,322 teaches methods and kits for determining olfactory perception. A test person's olfactory perception is evaluated and then determined by first providing the test subject with a palette of varying odors and fragrances, and then having that person describe, in full detail, each scent sample.

US 2019156224 teaches a method for predicting olfactory perception.

A library including a plurality of indexed olfactory descriptions as well as olfactory target description are received. Based on them a coefficient matrix and a perceptual distance between an indexed olfactory description and an olfactory target description is calculated. The method generates a perceptual description prediction for the olfactory target.

US201807803 describes a method for correlating molecular structure with olfactory perception.

None of the above mentioned documents combines olfactory prediction of an aroma molecule based on chemical structure with odorant descriptions obtained by human olfactory evaluation. Use of this comparison for quality assessment purposes is not disclosed.

There is a need in the art for new quality assessment processes to render the assessment process simpler, faster, cheaper, and more sustainable.

Analytical methods are time consuming and expensive. A problem associated therewith, is that usually perfume producers and raw material as well as intermediate suppliers are not equipped and employees are not qualified for sample testing. Therefore, it is desirable that the quality of an aroma molecule is predictable without use of analytical methods.

There is, thus, a need in the art to provide reliable means and methods for quality assessment of aroma molecules. In particular, there is a need to provide means and methods avoiding at least in part the drawbacks of the prior art as discussed above.

This problem is solved by the methods, apparatus, system, and uses with the features of the independent claims. Preferred embodiments, which might be realized in an isolated fashion or in any arbitrary combination are listed in the dependent claims.

Accordingly, the present invention relates to a computer-implemented method for quality assessment of aroma molecules using single-molecule olfactory predictions comprising
(i) receiving input via an input unit of at least one set of odorant descriptions (A) obtained by olfactory prediction of an aroma molecule and a second set of odorant descriptions (B) obtained by human olfactory evaluation; (ii) comparing the received set of odorant descriptions (A) obtained by olfactory prediction of the aroma molecule to the second set of odorant descriptions (B) obtained by olfactory prediction of the same aroma molecule; (iii) determining, specifically calculating, quality of aroma molecule based on the discrepancy between the at least one set of odorant descriptions (A) obtained by olfactory prediction of the aroma molecule and the second set of odorant descriptions (B) obtained by human olfactory evaluation via a processing unit (20); and
(iv) providing, preferably via an output unit (30), quality assessment results of the aroma molecule.

The method of the present invention may comprise steps in addition to those explicitly mentioned above.

Moreover, the method may be preceded by steps establishing a model of olfactory predictions, e.g. by a method for providing an olfactory model for an aroma molecule. Moreover, the data from the olfactory model may be provided in a database, preferably tangibly embedded into a data carrier, comprising an identification code for at least one aroma molecule and, allocated thereto, at least the parameters required for determining a set of odorant descriptions of the aroma molecule. As will be understood by the skilled person, the aforesaid method for providing a olfactory model preferably precedes the computer-implemented method for quality assessment of aroma molecules and, also preferably, is performed only once to establish the model and, preferably, include the required parameters into the aforesaid database.

Referring to the computer-implemented aspects of the invention, one or more of the method steps, preferably all of the method steps of the method according to one or more of the embodiments disclosed herein may be performed by using a computer or computer network. Thus, generally, any of the method steps including provision and/or manipulation of data may be performed by using a computer or computer network. Generally, these method steps may include any of the method steps, typically except for method steps requiring manual work, such as providing the samples and/or certain aspects of performing the human olfactory evaluation.

Specifically, further disclosed herein are:
a computer or computer network comprising at least one processor, wherein the processor is adapted to perform the method according to one of the embodiments described in this description,
a computer loadable data structure that is adapted to perform the method according to one of the embodiments described in this description while the data structure is being executed on a computer,
a computer program, wherein the computer program is adapted to perform the method according to one of the embodiments described in this description while the program is being executed on a computer,
a computer program comprising program means for performing the method according to one of the embodiments described in this description while the computer program is being executed on a computer or on a computer network,
a computer program comprising program means according to the preceding embodiment, wherein the program means are stored on a storage medium readable to a computer,
a storage medium, wherein a data structure is stored on the storage medium and wherein the data structure is adapted to perform the method according to one of the embodiments described in this description after having been loaded into a main and/or working storage of a computer or of a computer network, and
a computer program product having program code means, wherein the program code means can be stored or are stored on a storage medium, for performing the method according to one of the embodiments described in this description, if the program code means are executed on a computer or on a computer network.

The devices and methods according to the present invention have several advantages over known methods for quality assessment of aroma molecules. The use of a computer-implemented method may allow to render the assessment process simpler, faster, cheaper, and more sustainable.

As used in the following, the terms “have”, “comprise” or “include” or any arbitrary grammatical variations thereof are used in a non-exclusive way. Thus, these terms may both refer to a situation in which, besides the feature introduced by these terms, no further features are present in the entity described in this context and to a situation in which one or more further features are present. As an example, the expressions “A has B”, “A comprises B” and “A includes B” may both refer to a situation in which, besides B, no other element is present in A (i.e. a situation in which A solely and exclusively consists of B) and to a situation in which, besides B, one or more further elements are present in entity A, such as element C, elements C and D or even further elements.

Further, as used in the following, the terms “preferably”, “more preferably”, “most preferably”, “particularly”, “more particularly”, “specifically”, “more specifically” or similar terms are used in conjunction with optional features, without restricting further possibilities. Thus, features introduced by these terms are optional features and are not intended to restrict the scope of the claims in any way. The invention may, as the skilled person will recognize, be performed by using alternative features. Similarly, features introduced by “in an embodiment” or similar expressions are intended to be optional features, without any restriction regarding further embodiments of the invention, without any restrictions regarding the scope of the invention and without any restriction regarding the possibility of combining the features introduced in such way with other optional or non-optional features of the invention.

As used herein, if not otherwise indicated, the term “about” relates to the indicated value with the commonly accepted technical precision in the relevant field, preferably relates to the indicated value+20%, more preferably +10%, most preferably +5%. Further, the term “essentially” indicates that deviations having influence on the indicated result or use are absent, i.e. potential deviations do not cause the indicated result to deviate by more than +20%, more preferably +10%, most preferably +5%. Thus, “consisting essentially of” means including the components specified but excluding other components except for materials present as impurities, unavoidable materials present as a result of processes used to provide the components, and components added for a purpose other than achieving the technical effect of the invention. For example, a composition defined using the phrase “consisting essentially of” encompasses any known acceptable additive, excipient, diluent, carrier, and the like. Preferably, a composition consisting essentially of a set of components will comprise less than 5% by weight, more preferably less than 3% by weight, even more preferably less than 1%, most preferably less than 0.1% by weight of non-specified component(s).

The term “odorant description”, as used herein, includes, without limitation, any type of odorant character (or quality) which is a nominal measurement scale (categories). For the characterization of odour, a reference vocabulary for taste and odour sensation is used. Amongst the most common forms of representing odour is the Odour Description Wheel, proposed by McGinley & McGinley (2002), Odor testing biosolids for decision making. In: Water Environment Federation Specialty Conference. Residuals and Biosolids Management Conference. Austin (EUA), 3-6., in which eight odour categories families are easily recognized (see FIG. 2) or the ScenTree by ScenTree SAS in Paris, France (see https://www.scentree.co/). From that, the number (or percentage) of responses can be represented in the form of a histogram or graph.

For perfume fragrance compositions the odour descriptions of the aroma molecules are usually categorized in about 40 main descriptions and 500 detailed descriptions such as e.g. fruity, green, fresh, floral, woody or spicy as main descriptions and e.g. apple, lemon, citrus, strawberry, cherry or grapes as descriptions of fruity.

The term “aroma molecule”, as used herein, includes, without limitation, any type of molecule that has a smell or odor.

For an individual aroma molecule or class of aroma molecules to impart a smell or fragrance, it preferably is sufficiently volatile for transmission via the air to the olfactory system in the upper part of the nose. As examples, various fragrant fruits have diverse aroma molecules, particularly strawberries which are commercially cultivated to have appealing aromas, and contain several hundred aroma molecules.

Preferably, aroma molecules have molecular weights of less than 310 g/mol. A flavor denotes an aroma chemical which induces a taste impression. A “fragrance” or “scent” denotes an aroma chemical which induces a odor impression. Flavors tend to be naturally occurring, and the term fragrances may also apply to synthetic compounds, such as those used in cosmetics.

Aroma molecules can be found in various foods, such as fruits and their peels, wine, spices, floral scent, perfumes, fragrance oils, and essential oils. For example, many form biochemically during the ripening of fruits and other crops. Wines have more than 100 aromas that form as byproducts of fermentation. Also, many of the aroma molecules play a significant role in the production of compounds used in the food service industry to flavor, improve, and generally increase the appeal of their products.

Preferably aroma molecules are esters, carboxylic acids, linear terpenes, cyclic terpenes, aromatic compounds, amines, nitriles, pyrroles, pyrazines, thiazoles, alcohols, acetals, acyclic and cyclic ethers, aldehydes, ketones, lactones, thiols, and hydrocarbons. More preferably aroma molecules are esters, linear terpenes, cyclic terpenes, aromatic compounds and amines.

Preferred esters are geranyl acetate, methyl formate, methyl acetate, methyl propionate, methyl butyrate, ethyl acetate, ethyl butyrate, isoamyl acetate, pentyl butyrate, pentyl pentanoate, octyl acetate, benzyl acetate, methyl anthranilate, hexyl acetate, fructone, ethyl methylphenylglycidate and alpha-methylbenzyl acetate.

7 PCT/EP2022/067251 WO 2023/274850 Preferred carboxylic acids are 2-methyl-2-pentenoic acid, and phenylacetic acid.

Preferred linear terpenes are myrcene, geraniol, nerol, citral, citronellal, citronellol, linalool, nerolidol and ocimene.

Preferred cyclic terpenes are limonene, camphor, menthol, carvone, terpineol, alpha-ionene, thujone, eucalyptol and jasmone.

Preferred aromatic compounds are benzaldehyde, eugenol, cinnamaldehyde, ethyl maltol, vanillin, anisole, anethole, estragole and thymol.

Preferred amines are trimethylamine, putrescine, cadaverine, pyridine, indole and skatole.

Preferred nitriles are geranylnitrile, and 2-tridecenenitrile.

Preferred pyrroles are 2-acetylpyrrole, and 2-acetyl-3,4-dihydro-5H-pyrrole.

Preferred pyrazines are 2-acetylpyrazine, and 2-methylthio-3-methylpyrazine.

Preferred thiazoles are 2-isobutylthiazole, and 2,5-dimethylthiazole.

Preferred alcohols are furaneol, 1-hexanol, cis-3-hexen-1-ol and menthol.

Preferred acetals are citral diethyl acetal, acetaldehyde ethyl phenethyl acetal, and hyroxydihydrocitronellal dimethyl acetal.

Preferred ethers are phenethyl isoamyl ether, and cyclododecyl methyl ether.

Preferred aldehydes are acetaldehyde, hexanal, cis-3-hexanal, furfural, hexyl cinnamaldehyde, isovaleraldehyde, anistic aldehyde, cuminaldehyde.

Preferred ketones are cyclopentadecanone, dihydrojasmone, oct-1-en-3-one, 2-acetyl-1-pyrroline and 6-acetyl-2,3,4,5-tetrahydropyridine.

Preferred lactones are gamma-decalactone, gamma-nonalactone, delta-octolactone, jasmine lactone, massoia lactone, wine lactone and sotolon.

Preferred thiols are thioacetone, allyl thiol, ethanethiol, 2-methyl-2-propanethiol, butane-1-thiol, grapefruit mercaptan, methanethiol, furan-2-ylmethanethiol and benzyl mercaptan.

Preferred hydrocarbons are 1,3,5-undecatriene, and 1,3-undecadien-5-yne.

The aroma molecules might be part of an aroma molecule formulation.

The odorant descriptions (A) obtained by olfactory prediction of an aroma molecule, can be obtained from a database or preferably predicted using machine learning algorithms. The descriptions that are common for different aroma molecules are bundled in odour main families and optionally odour subfamilies.

Such databases can be commercially available databases such as e.g. ScenTree, Flavornet, GoodScents, SuperScent or Sigma-Aldrich or an internal database.

Such a database usually lists the name and the structure of the aroma molecule together with several descriptions of the main and sub-odour families and optional additional attributes of the aroma molecule, such as e.g. molecular weight or boiling points, if available. Usually the number of descriptions of the aroma molecule varies from about 4 to 10.

The descriptions of the odour families of said aroma molecule can also be predicted using machine learning algorithms, e.g. in the case that the descriptions of the odour families of a specific aroma molecule is not available from a database. These algorithms are preferably trained on a computer-readable 2D or 3D description of the molecular structure to identify lead structures in the molecular structure of the aroma molecule, which contribute to the olfactive perception of the aroma molecule.

Preferred algorithms are K-Nearest Neighbors, Naive Bayes, Linear Discriminant Analysis, Support Vector Machines, Logistic Regression, Neural Network and Random Forrest Algorithms. Very preferred are Random Forrest Algorithms.

K-Nearest Neighbors works by checking the distance from some test compounds to the known class labels of some training compounds. The group of compounds with their respective class labels that would deliver the smallest distance between the training compounds and the test compounds is the class label that the test compounds is assigned to.

Naive Bayes Classifiers determine the probability that a compound belongs to a specific class by calculating the probability that an event will occur given that some input event has occurred. When applying this calculation, it is assumed that all class label predictions are independent.

Linear Discriminant Analysis operates by reducing the dimensionality of the dataset in detail by projecting all compounds onto a line. After this it combines these compounds into classes based on their distance from a chosen point or centroid.

Support Vector Machines (SVM) operate by drawing a line (decision boundary) between the different clusters of compounds to group them into classes. Compounds on one side of the decision boundary will be assigned to one class and compounds on the other will be assigned to another class.

Then SVMs will try to maximize the distance between the decision boundary and the compounds on either side of it. Dependent on which side the test compounds “fall”, they will be assigned to one or the other class respectively.

Logistic Regression outputs predictions of the test compounds on a binary scale (zero or one). In case the value of a compound is 0.5 or above, it is assigned to class 1 and if it is 0.5 is assigned to class 0.

A neural network consists of artificial input and output neurons. They are connected by weighted synapses. These weights influence the amount of forward propagation which goes through the neural network. The changing of the weights is done during the back propagation.

Then the neural network is “learning”.

A very preferred machine learning method is the random forest in the classification mode. A random forest is an ensemble of decision trees. The idea of ensemble building is, that many weak classifiers can be combined to a strong one. The final classifier is much less susceptible to overfitting to the training data. That leads to robust prediction models for unknown molecules. The molecules can be classified into two classes based on a so-called prediction score. The prediction score is a class probability estimator. Preferably, 0.5 is the decision boundary. In this case the two classes might be “smells like the certain class” or “smells not like the certain class”. These two classes are unequally distributed, that is why a random undersampling can be done. This technique samples the same number of molecules from the bigger class like from the smaller class. In addition, the decision boundary can be preferably moved to 0.7.

The prediction of the descriptions of the odour families of an aroma molecule using machine learning algorithms is preferably conducted in a computer-implemented method using a processing unit comprising one or more processors.

The aroma molecules are preferably grouped into odour families, whereby each aroma molecule is usually matched with 1 to 4, preferably 3 to 4 descriptions, which are either obtained from a database or predicted using machine learning algorithms. For example, the fragrance ingredient “ligustral” can be attributed with the descriptions green, herbaceous and citrus.

The descriptions can also be weighted with different factors of olfactive perception such as e.g. for the aroma molecule “ligustral” the olfactive perception of green is counted to be 1, the olfactive perception of herbaceous is counted to be 0.4 and the olfactive perception of citrus is counted to be 0.2.

The olfactory prediction of an aroma molecule is preferably performed by a method comprising:
receiving, by the processor, the chemical structure of the aroma molecule;
calculating, by the processor, based on the chemical structure of the aroma molecule odorant descriptors; and
generating the olfactory prediction for the aroma molecule.

The prediction of odorant descriptions using machine learning algorithms is preferably based on molecular descriptors.

Molecular descriptors can include chemical information, such as chemical formulas, structures, substructures, and physical properties. Molecular descriptors can be included in chemoinformatic feature vectors that summarize the chemical and/or molecular properties of a substance.

Theoretical molecular descriptors can include 0D-descriptors, 1D-descriptors, 2D-descriptors, 3D-descriptors, and 4-D descriptors. 0D-descriptors can include, for example, constitutional descriptors that describe the arrangement of elements within a molecule, the types of chemical bonds present in the molecule, etc. 0D-descriptors can further include count descriptors that indicate, for example, the number of atoms of each element present in a molecular compound. 1D-descriptors can include, for example, lists of structural fragments, fingerprints, or the like. 2D-descriptors can include, for example, graph invariants or the like. 3D-descriptors can include, for example, 3D-Molecule Representation of Structure based on Electron Diffraction (MORSE) descriptors; Weighted Holistic Invariant Molecular (WHIM) descriptors; Geometry, Topology, and Atom Weights Assembly (GETAWAY) descriptors; quantum-chemical descriptors; size, steric, surface, and volume descriptors; or the like. 4D-descriptors can include, for example, Grid-Independent descriptors (GRIND) or descriptors obtained through Comparative Molecular Field Analysis (CoMFA) methods. The set of molecular descriptors that is used can include any combination of the above-described types of molecular descriptors. Further, in certain example embodiments, a descriptor must satisfy various criteria in order to be used. For example, a molecular descriptor can need to be invariant to molecular numbering or labeling.

Preferred descriptors are physicochemical descriptors, topological descriptors, EVA descriptors, and/or Morgan fingerprint descriptors.

For providing the physicochemical descriptors to the molecule data, the RDKit extension in KNIME can be used. Based on the provided SMILES strings, RDKit can determine the physicochemical properties of the molecule, e.g. the molecular weight and the log(Pow) value but also can count the number of molecular features, e.g. the number of unsaturated carbon rings and number of halogenic atoms in the molecule. E.g. more than 100 descriptors can be added to each molecule.

Morgan fingerprint descriptors encode local structural information of a molecule with respect to its radial neighboring atoms, as sketched in FIG. 4. As a starting point, usually every heavy atom is taken into account where its atomic information is noted. The “Extended Connectivity Fingerprint” (ECFP) is based on the Morgan algorithm. It collects in the initial step the information of the atomic number of the atom and its hybridization state which is called the “initial assignment stage”. Afterwards, in the “iterative updating stage” the neighbors of each atom are taken into account and in the final “duplicate identifier removal” stage, identical features are reduced to a single representative bit string. This iteration is proceeded until all atom identifiers are as unique as the molecular symmetry allows it. For the characterization of each atom, in theory, any rule that generates values for the observed atom could be used.

The EVA descriptor has already been introduced by Shell in the 1990s (A. M. Ferguson, T. Heritage, P. Jonathon, S. E. Pack, L. Phillips, J. Rogan, P. J. Snaith, J. Comp. Aided Mol. Des., 1997, 11, 143-152), as a robust descriptor for the molecular structure in QSPR/QSAR predictions, e.g., for predicting the octanol-water partition coefficient. It is derived from quantum chemically computed normal coordinate frequencies, hence termed EigenVAlue (EVA) descriptor.

Once the frequencies are determined they are projected onto a linear bound frequency scale, typically in the range of 1-4000 cm 1. This range should include all fundamental vibrational normal modes. Next, a Gaussian kernel of fixed width (o) is centered at each frequency value. The linear bound frequency scale is then sampled at fixed increments of L cm 1 and the value of the resulting EVA descriptor EVAx at each sample point x is the sum of the amplitudes of the overlaid kernels at that point

$${EVA}_{r} = {\sum }_{i = 1}^{3 N - 6} \frac{1}{σ \sqrt{2 π}} {e}^{- \frac{{( x - fi )}^{2}}{2 {σ}^{2}}}$$

where fᵢ is the i-th normal mode frequency of the compound.

The EVA descriptors for an aroma molecule can be computed according to the following steps:
1. Generate the 3D structure (if not yet available) from a chemical identifier such as SMILES or InChi via e.g. RDkit, OpenBabel, ChemAxon, Maestro, Avogadro or any other (GUI based) molecule editor. In case the stereochemistry is not fully defined, generate 3D structures for all possible stereoisomers. It is preferred using RDKit to convert a SMILES to a 3D structure.
2. For each stereoisomer either generate a conformers ensemble with software like Balloon, CORINA, ConfGen, Frog2, MOE, or RDKit (e.g. via the EDKDG algorithm), or obtain a conformers ensemble from a molecular dynamics simulation with classical force fields or quantum chemical methods, i.e., semi-empirical methods (e.g. XTB, DFTB, PM6, or AM1), density functional theory based methods (e.g. PBE, B3LYP, or TPSS), or wave function based methods (e.g. HF or MP2). It is preferred running an XTB meta-dynamics simulation with CREST using the iMTD-GC algorithm.
3. For each conformer compute the vibrational frequencies using a semi-empirical quantum chemical method (e.g. XTB, DFTB, PM6, or AM1), a density functional theory based method (e.g. PBE, TPSS, or B3LYP), or a wave function based quantum chemical method (e.g. HF or MP2) either in vacuum or employing an implicit solvation model like COSMO or PCM. It is preferred using the semi-empirical method XTB in combination with the GBSA solvent model.
4. Compute the EVA descriptor for the lowest energy conformer, see equation above.
5. Compute the EVA descriptor for the conformer ensemble and weight contributions according to Boltzmann factors (neglect the conformer if its Boltzmann factor is small, e.g. below 0.001):

$${EVA}_{x} \begin{matrix} \\ =\end{matrix} {\sum }_{j} ( - \frac{Δ E}{RT} ) {EVA}_{x}^{j}$$

The term “aroma molecule formulation”, as used herein, includes, without limitation, any type of formulation comprising at least one aroma molecule. Thus, the formulation may be liquid or solid, may be a solution, an emulsion, a suspension, a sol, a gel, or a solid. Preferably, the formulation is a solution, an emulsion, or a suspension, more preferably is a solution. Preferably, the formulation comprises additional compounds in addition to the aroma molecule, in particular other aroma molecules, buffer compounds, salts, stabilizers, solvents, and the like.

Also preferably, the aroma molecule formulation is an aroma molecule solution, preferably an aqueous solution. Thus, preferably, the aroma molecule formulation further comprises water. The aroma molecule formulation may comprise more than one aroma molecule. Preferably, in such case, the quality assessments are based on the aroma molecule showing the strongest smell or odor.

To obtain the set of odorant descriptions B by human olfactory evaluation the human subject is presented with a set of odorant descriptions for each sniffed aroma molecule. The odorant descriptions are optionally and preferably presented by a user interface such as, but not limited to, a graphical user interface displayed on a computer screen, a smart TV screen, or a screen of a mobile device, e.g., a smartphone device, a tablet device or a smartwatch device. In some embodiments, a set of rating controls is also displayed, preferably on the same screen.

The odorant descriptions are human-language descriptions and are presented in a human-readable form to allow the subject to read and decipher them. Preferably, each of the descriptions is associated with a known odor, not necessarily odor that is emitted by one of the aroma molecules, or a subjective perception of odor. For example, a description can be a textual phrase, such as, but not limited to, “smells like coconut” or “smells like rubber” or “does not smell like gasoline” or “has a pleasant smell” or “has an unpleasant smell” or the like. The subject may also provide additional odorant descriptions next to the presented odorant descriptions. Preferably the descriptions of the aroma molecules can be categorized in certain number of main descriptions and a higher number of subdescriptions such as e.g. fruity, green, fresh, floral, woody or spicy as main descriptions and e.g. apple, lemon, citrus, strawberry, cherry or grapes as subdescriptions of fruity.

The rating controls that are displayed can be of any type generally known in the field of graphical user interface design. Representative examples include, without limitation, a slider, a dropdown menu, a combo box, a text box and the like. A representative set of human-language descriptions with a respective set of rating controls is illustrated in FIG. 3.

In embodiments in which rating controls are displayed, the user enters the ratings in the rating controls, and the ratings are received from the rating controls. Each received rating is indicative of a descriptiveness of the respective odorant description for the respective aroma molecule, as perceived by the human subject upon sniffing that odorant sample. For example, when the odorant description is “has a pleasant smell,” the sniffing rating indicates to what extent the subject perceives the pleasantness of the odor of the respective odorant.

The descriptiveness levels are preferably numerical according to a predetermined scale, for example, 0 to 100. The ratings, on the other hand, are not necessarily numerical. For example, the ratings can be positions on a slider or textual phrases from a dropdown menu. In embodiments in which the ratings are not numerical, the method optionally and preferably parses the ratings and maps them to numerical descriptiveness levels according to a predetermined mapping protocol. It is appreciated, however, that some subjects may not provide a rating for each and every odorant description that is displayed, since, for example, some subjects may find a particular odorant description irrelevant for a particular aroma molecule.

In certain example embodiments, the aroma molecules can be presented to one or more human subjects in different concentrations, and the human subjects can rate different sets of descriptions with respect to the different concentrations. For example, human suspects can be asked to rate the “high” concentration molecular samples with respect to selected descriptions and rate the “low” concentration molecular samples with respect to other descriptions. Alternatively, all concentrations of the aroma molecule samples can be rated with respect to each description. In certain example embodiments, the ratings can be normalized across an aggregate number of individuals. Further, in certain example embodiments, the ratings can be aggregated across multiple individuals and a statistical measure (e.g., mean, median, etc.) can be generated to reflect aggregate human olfactory evaluation for the aroma molecule.

The term “input data” as used herein is a broad term and is to be given its ordinary and customary meaning to a person of ordinary skill in the art and is not to be limited to a special or customized meaning. The term preferably refers to at least one set of odorant descriptions (A) obtained by olfactory prediction of an aroma molecule and a second set of odorant descriptions (B) obtained by human olfactory evaluation. Preferably, input data comprises further data, preferably data weighting the descriptions with different factors of olfactive perception.

The term “input unit”, as used herein, includes without limitation any item or element forming a boundary configured for transferring information. In particular, the input unit may be configured for transferring information onto a computational device, e.g. onto a computer, such as to receive information. The input unit preferably is a separate unit configured for receiving or transferring information onto a computational device, e.g. one or more of: an interface, specifically a web interface and/or a data interface; a keyboard; a terminal; a touchscreen, or any other input device deemed appropriate by the skilled person. More preferably, the input unit comprises or is a data interface configured for transferring or exchanging information as specified herein below.

The term “output unit”, as used herein, includes without limitation any item or element forming a boundary configured for transferring information. In particular, the output unit may be configured for transferring information from a computational device, e.g. a computer, such as to send or output information, e.g. onto another device or to a user. The output unit preferably is a separate unit configured for outputting or transferring information from a computational device, e.g. one or more of: an interface, specifically a web interface and/or a data interface; a screen, a printer, or a touchscreen, or any other output device deemed appropriate by the skilled person. More preferably, the output unit comprises or is a data interface configured for transferring or exchanging information as specified herein below. The output unit preferably provides quality assessment results of the aroma molecule.

Preferably the quality assessment results contain information about contamination by chemical or physical degradation of the aroma molecule.

Also preferably the quality assessment results contain information about contamination of the aroma molecule from external source.

Also preferably the quality assessment results contain information about structural isomers and/or side product(s) from synthesis of the aroma molecule.

Preferably, the input unit and the output unit are configured as at least one or at least two separate data interface(s); i.e. preferably, provide a data transfer connection, e.g. a wireless transfer, an internet transfer, Bluetooth, NFC, inductive coupling or the like. As an example, the data transfer connection may be or may comprise at least one port comprising one or more of a network or internet port, a USB-port and a disk drive. The input unit and/or the output unit may also be may be at least one web interface.

The term “processing unit” as used herein is a broad term and is to be given its ordinary and customary meaning to a person of ordinary skill in the art and is not to be limited to a special or customized meaning. The term specifically may refer, without limitation, to an arbitrary logic circuitry configured for performing operations of a computer or system, and/or, generally, to a device or unit thereof which is configured for performing calculations or logic operations. The processing unit may comprise at least one processor. In particular, the processing unit may be configured for processing basic instructions that drive the computer or system. As an example, the processing unit may comprise at least one arithmetic logic unit (ALU), at least one floating-point unit (FPU), such as a math coprocessor or a numeric coprocessor, a plurality of registers and a memory, such as a cache memory. In particular, the processing unit may be a multi-core processor. The processing unit may comprise a Central Processing Unit (CPU) and/or one or more Graphics Processing Units (GPUs) and/or one or more Application Specific Integrated Circuits (ASICs) and/or one or more Tensor Processing Units (TPUs) and/or one or more field-programmable gate arrays (FPGAs) or the like. The processing unit may be configured for pre-processing the input data. The pre-processing may comprise at least one filtering process for input data fulfilling at least one quality criterion. For example, the input data may be filtered to remove missing variables. Preferably, input data may be compared to at least one pre-defined threshold value, e.g. a threshold number of odorant descriptions, to determine whether method step (ii) is required to be performed at all. Preferably, the processing unit is configured to perform a determination, preferably calculation, of a quality of an aroma molecule based on the discrepancy between the at least one set of odorant descriptions (A) obtained by olfactory prediction of the aroma molecule and the second set of odorant descriptions (B) obtained by human olfactory evaluation. Methods for determining a quality of aroma molecule based on the discrepancy between the at least one set of odorant descriptions (A) obtained by olfactory prediction of the aroma molecule and the second set of odorant descriptions (B) obtained by human olfactory evaluation are specified elsewhere herein. Preferably, said determining is based on previously established data on possible contamination of the aroma molecule in question. Preferably, the data contains information on contamination by chemical or physical degradation of the aroma molecule. The data can also preferably contain information about contamination from external source or about structural isomers of the aroma molecule and/or side product(s) from synthesis of the aroma molecule. More preferably olfactory information of the e.g. degradation products are compared to the discrepancy between the set of odorant descriptions (A) obtained by olfactory prediction of the aroma molecule and the second set of odorant descriptions (B) obtained by human olfactory evaluation.

The definitions made above apply mutatis mutandis to the following. Additional definitions and explanations made further below also apply for all embodiments described in this specification mutatis mutandis.

The present invention further relates to an apparatus for quality assessment of aroma molecules using single-molecule olfactory predictions comprising:
an input unit (10) configured to receive a data input, preferably a user interface, wherein the data input comprises at least one set of odorant descriptions (A) obtained by olfactory prediction of an aroma molecule and a second set of odorant descriptions (B) obtained by human olfactory evaluation;
a processing unit (20), preferably a processing unit comprising at least one processor, configured, specifically by programming, to compare the received set of odorant descriptions (A) obtained by olfactory prediction of an aroma molecule to the second set of odorant descriptions (B) obtained by olfactory prediction of the same aroma molecule and determine, specifically to calculate, the quality of aroma molecule based on the discrepancy between the at least one set of odorant descriptions (A) obtained by olfactory prediction of the aroma molecule and the second set of odorant descriptions (B) obtained by human olfactory evaluation; and an output unit (30) configured to output the quality assessment results of the aroma molecule to the user and/or to a data interface.

The term “apparatus”, as used herein, relates to a system of means comprising at least the aforementioned means operatively linked to each other as to allow the determination. Typical input and output units and means for carrying out the determination, in particular processing units, are disclosed above in connection with the methods of the invention. How to link the means in an operating manner will depend on the type of means included into the device. The person skilled in the art will realize how to link the means without further ado. Preferably, the means are comprised by a single apparatus. Typical apparatuses are those which can be applied without the particular knowledge of a specialized technician, in particular hand-held devices comprising an executable code, in particular an application, performing the determinations as specified elsewhere herein. The results may be given as output of raw data which need interpretation e.g. by a technician. More preferably, the output of the apparatus is, however, processed, i.e. evaluated, raw data, the interpretation of which does not require a technician. Also preferably, some functions of quality assessment of aroma molecules may be performed automatically, i.e. preferably without user interaction, e.g. adjustment of a synthesis of the aroma molecule. Further typical devices comprise the units, in particular the input unit, the processing unit, and the output unit referred to above in accordance with the method of the invention.

The input unit of the device may be configured to retrieve input data from a local storage device, e.g. a USB storage device or a sensor having stored storage segment data during storage and/or transport. The input device may, however, also receive input data from an external data storage means, e.g. via a data connection such as the internet.

The apparatus preferably is a handheld device or any type of computing device having the features as specified.

In addition to the quality assessment measures as specified herein above, the apparatus preferably is configured to further perform at least one of:
download relevant information including quality information, regulatory information, safety data, and/or technical documents;
and/or
provide a user-feedback including usability and/or information content.

The present invention also relates to a system for quality assessment of aroma molecules using single-molecule olfactory predictions, comprising:
an apparatus according to the present invention; and
a web server configured to interface with a user via a webpage served by the web server and/or an application program;
wherein the system is configured to provide a graphical user interface (GUI) to a user by the webpage and/or the application program.

The term “system” as used herein is a broad term and is to be given its ordinary and customary meaning to a person of ordinary skill in the art and is not to be limited to a special or customized meaning. The term includes, without limitation, any a setup having at least two interacting components. Specifically, the term may include any type of system comprising the components as specified. Preferably, the apparatus comprised in the system is an apparatus as specified herein above. Preferably, the apparatus is a computing device comprising a data interface as an input unit and as an output unit. Thus, preferably, the apparatus comprised in the system preferably is configured to receive input data from an external data storage means, e.g. via a data connection such as the internet.

The system is configured to output quality assessment results of the aroma molecule to an external data storage means and/or processing device, preferably a handheld device or remote computing device, via a web server configured to interface with a user via a webpage served by the web server and/or via an application program, wherein the system is configured to provide a graphical user interface (GUI) to a user by the webpage and/or the application program. Thus, preferably, the server is configured to provide a graphical user interface (GUI) to a user by the webpage and/or the application program. The term “graphical user interface” is known to the skilled person to relate to a user interface allowing a user to interact with an electronic device, in particular an apparatus or other computing device, through visual indicators instead of text-based user interaction, such as typed commands or text navigation. Also the term “application program” abbreviated as “application” or “App”, is also known to the skilled person as a computer executable code, in particular a software program providing a graphical user interface for a computing device function or a specific application of a computing device. Preferably, the application program is an executable code opening the web page served by the apparatus as specified elsewhere herein, preferably on a handheld device.

As the skilled person will understand in view of the present description, the web server may serve the quality assessment results of the aroma molecule as such; the web server may, however, also provide all parameters required to determine quality assessment results.

Thus, the web server, preferably, serves to a user at least one of
information about contamination by chemical or physical degradation of the aroma molecule;
information about contamination from external source; and
information about structural isomers and/or side product(s) from synthesis.

The present invention also relates to a computer program comprising instructions which, when the program is executed by the apparatus of the present invention, specifically by a processor of the apparatus, and/or by the system of the present invention, cause the apparatus and/or the system to perform the method of the present invention.

The present invention also relates to a computer-readable storage medium comprising instructions which, when executed by the apparatus of any one of the present invention and/or the system of any one of the present invention, cause the apparatus and/or the system to perform the method of the present invention.

As used herein, the terms “computer-readable data carrier” and “computer-readable storage medium” specifically may refer to non-transitory data storage means, such as a hardware storage medium having stored thereon computer-executable instructions. The computer-readable data carrier or storage medium specifically may be or may comprise a storage medium such as a random-access memory (RAM) and/or a read-only memory (ROM).

The present invention also relates to a use of a computer-implemented method according to the present invention and/or quality assessment results of the aroma molecule determined according to the method according the present invention in a chemical production process of an aroma molecule, preferably for determining production parameters; and to a use of a computer-implemented method according to the present invention in quality control processes.

The present invention further relates to a method for producing an aroma molecule having pre-defined quality assessment results, comprising the steps of the method for quality assessment of aroma molecules of the present invention and the further step of automatically adjusting production parameters in chemical production processes of an aroma molecule based on the quality assessment results of the aroma molecule.

The term “product comprising an aroma molecule” as used herein is a broad term and is to be given its ordinary and customary meaning to a person of ordinary skill in the art and is not to be limited to a special or customized meaning. In particular, the term includes any type of product comprising an aroma molecule preferably comprising a pre-defined quality assessment result.

Preferably, the product is a product for use in olfactory and taste applications, more preferably is a perfume or component thereof.

All references cited in this specification are herewith incorporated by reference with respect to their entire disclosure content and the disclosure content specifically mentioned in this specification.

### FIGURE LEGENDS

FIG. 1:

Device/system

FIG. 2;

FIG. 2 shows the Odour Description Wheel, proposed by McGinley & McGinley (2002), Odor testing biosolids for decision making. In: Water Environment Federation Specialty Conference. Residuals and Biosolids Management Conference. Austin (EUA), 3-6 as one example of an odour description wheel showing odour description main and subclasses only for illustrating purposes. Said distinct odour description wheel is shown here as illustrative example as it only includes a limited number of main and subclasses but still gives an impression on how main and subclasses are selected.

FIG. 3:

FIG. 3 shows a representative set of human-language descriptions with a respective set of rating controls.

FIG. 4:

FIG. 4 shows Morgan fingerprint descriptors that encode local structural information of a molecule with respect to its radial neighboring atoms.

FIG. 5:

Decomposition of aroma molecule agrumex.

FIG. 6:

Contamination in aroma molecule α-Damascone.

The following Examples shall merely illustrate the invention. They shall not be construed, whatsoever, to limit the scope of the invention.

### EXAMPLE 1

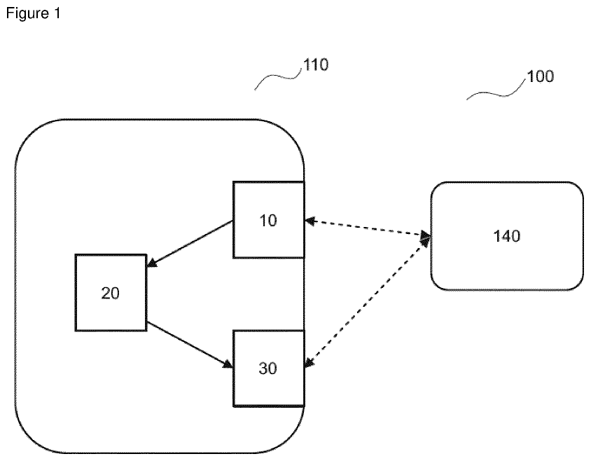

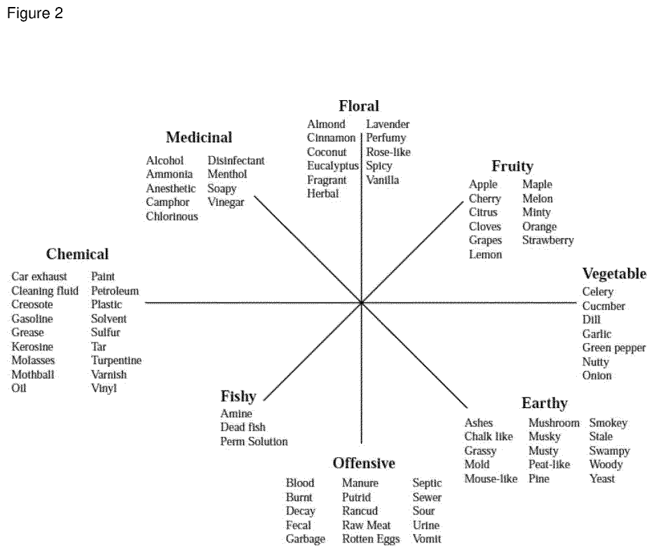

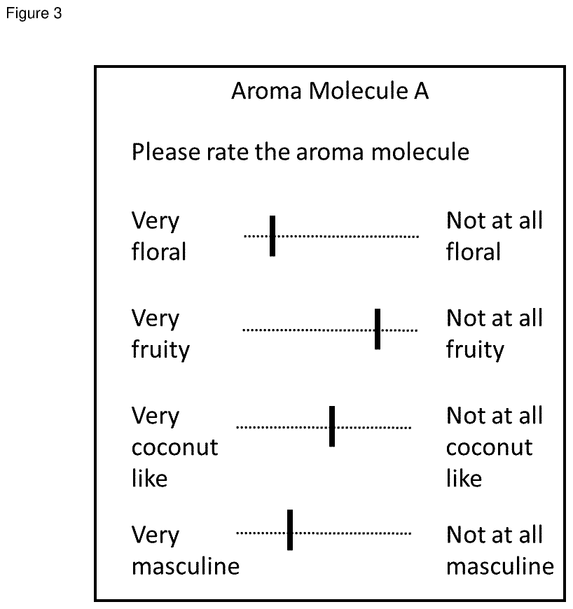

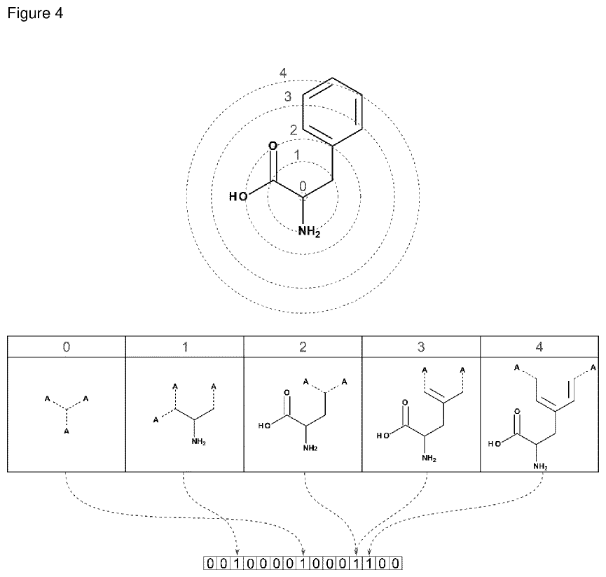

As shown in FIG. 1, a system 100 for quality assessment of aroma molecules using single-molecule olfactory predictions is disclosed. The system 100 comprises an apparatus 110 for quality assessment of aroma molecules and, further, a web server 140 configured to interface with a user via a webpage served by the web server and/or via an application program. The apparatus 110 comprises an input unit 10, a processing unit 20, and an output unit 30. In the system 100, the web server 140 may communicate with the input unit 10 and/or the output unit 30.

Apparatus 110 comprises at least one processing unit 20 such as a processor, microprocessor, or computer system, in particular for executing a logic in a given algorithm. The apparatus 110 may be configured for performing and/or executing at least one computer program of the present description. The processing unit 30 may comprise at least one processor. In particular, the processing unit 30 may be configured for processing basic instructions that drive the computer or system. As an example, the processing unit 30 may comprise at least one arithmetic logic unit (ALU), at least one floating-point unit (FPU), such as a math co-processor or a numeric coprocessor, a plurality of registers and a memory, such as a cache memory. In particular, the processing unit 30 may be a multi-core processor. The processing unit 30 may be configured for machine learning. The processing unit 30 may comprise a Central Processing Unit (CPU) and/or one or more Graphics Processing Units (GPUs) and/or one or more Application Specific Integrated Circuits (ASICs) and/or one or more Tensor Processing Units (TPUs) and/or one or more field-programmable gate arrays (FPGAs) or the like.

The apparatus comprises at least one communication interface, preferably an output unit 30, configured for outputting data. The communication interface may be configured for transferring information from a computational device, e.g. a computer, such as to send or output information, e.g. onto another device. Additionally or alternatively, the communication interface may be configured for transferring information onto a computational device, e.g. onto a computer, such as to receive information, i.e. may be an input unit 10. The communication interface may specifically provide means for transferring or exchanging information. In particular, the communication interface may provide a data transfer connection, e.g. Blue-tooth, NFC, inductive coupling or the like. As an example, the communication interface may be or may comprise at least one port comprising one or more of a network or internet port, a USB-port and a disk drive. The communication interface may be at least one web interface. The input data comprises at least one set of odorant descriptions (A) obtained by olfactory prediction of an aroma molecule and a second set of odorant descriptions (B) obtained by human olfactory evaluation as specified herein above.

The processing unit 20 may be configured for pre-processing the input data. The pre-processing unit 20 may comprise at least one filtering process for input data fulfilling at least one quality criterion. The processing unit 20 is configured for determining quality of aroma molecule based on the discrepancy between the at least one set of odorant descriptions (A) obtained by olfactory prediction of the aroma molecule and the second set of odorant descriptions (B) obtained by human olfactory evaluation, preferably as specified herein above and below in the further Examples.

The web server 140 is configured to provide a GUI for the apparatus 110. Thus, the web server may exchange data with the output unit 30, e.g. for displaying said data on the GUI. The web server 140 may, however, also exchange data with the input unit of the apparatus.

### EXAMPLE 2

Decomposition of aroma molecule agrumex
secondary ester might eliminate acetic acid (HOAc)
decomposition products are an olefine and acetic acid, next to the unaltered product Agrumex

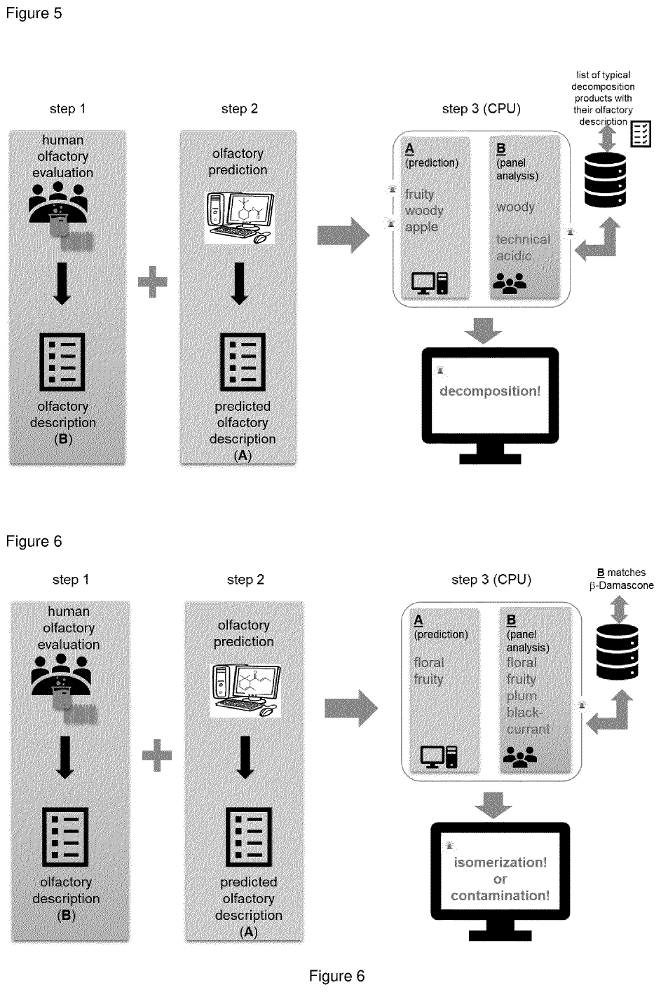

The received agrumex sample is subjected to human olfactory assessment by a panel of 4 persons. As a result of the panel's olfactory evaluation of the sample the odour of the sample is described as being woody, technical, and acidic. This result is stored in the system by the user by utilizing the GUI. In parallel, structural information in form of a SMILES code is provided to the program via the GUI and a single-molecule olfactory prediction is performed by the program. The result of the single-molecule olfactory prediction of agrumex is fruity, woody, and apple. The GUI displays description (A) consisting of the olfactory prediction of agrumex next to description (B) consisting of the human olfactory evaluation of the agrumex sample as depicted in FIG. 5. The program displays a warning stating that the sample is likely decomposed. Parts of the human olfactory description of the panel (technical and acidic) in combination with missing olfactory descriptions (fruity and apple) lead the program alert the user.

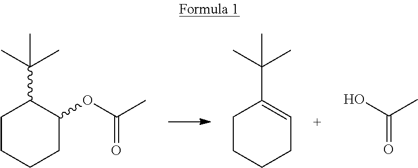

### EXAMPLE 3

Contamination in aroma molecule α-Damascone
isomerization of ring double bond
from a- to b-position
high degree of isomerization, due to possible acidic environment

This example demonstrates the detection of product isomerization towards another double bond isomer. The received a-Damascone sample is subjected to human olfactory assessment by a panel of 5 persons. As a result of the panel's olfactory evaluation of the sample the odor of the sample is described as being floral, fruity, plum, and blackcurrant. This result is stored in the system by the user by utilizing the GUI. In parallel, structural information in form of 2D structural drawing is provided to the program via the GUI and a single-molecule olfactory prediction is performed by the program. The result of the single-molecule olfactory prediction of a-Damascone is floral, and fruity. The GUI displays description (A) consisting of the olfactory prediction of a-Damascone next to description (B) consisting of the human olfactory evaluation of the a-Damascone sample as depicted in FIG. 6. The program displays a warning stating that the product sample is likely isomerized or contaminated. Parts of the human olfactory description of the panel (plum and blackcurrant) matched with a structure in the database that is very close to a-Damascone and, thus, lead the program alert the user. This similar fragrant is b-Damascone, a constitutional double bond isomer in the family of rose ketones.

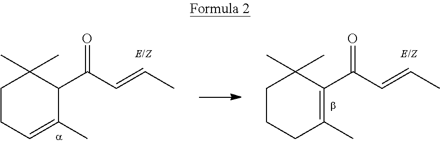

### REFERENCE SIGNS

10 input unit
20 processing unit
30 output unit
100 system
110 apparatus
140 web server

1. A computer-implemented method for quality assessment of aroma molecules using single-molecule olfactory predictions comprising:
(i) receiving input data, preferably via an input unit, of at least one set of odorant descriptions obtained by olfactory prediction of an aroma molecule and a second set of odorant descriptions obtained by human olfactory evaluation;
(ii) comparing the received set of odorant descriptions obtained by olfactory prediction of the aroma molecule to the second set of odorant descriptions obtained by olfactory prediction of the same aroma molecule;
(iii) determining quality of aroma molecule based on the discrepancy between the at least one set of odorant descriptions obtained by olfactory prediction of the aroma molecule and the second set of odorant descriptions obtained by human olfactory evaluation via a processing unit; and
(iv) providing, preferably via an output unit, quality assessment results of the aroma molecule.

2. The computer-implemented method of claim 1, wherein the quality assessment results contain information about contamination by chemical or physical degradation of the aroma molecule.

3. The computer-implemented method of claim 1, wherein the quality assessment results contain information about contamination from external source.

4. The computer-implemented method of claim 1, wherein the quality assessment results contain information about structural isomers and/or side product(s) from synthesis.

5. The computer-implemented method of claim 1, wherein the olfactory prediction of an aroma molecule is performed by a method comprising:
receiving, by the processor, the chemical structure of the aroma molecule;
calculating, by the processor, based on the chemical structure of the aroma molecule odorant descriptors; and
generating the olfactory prediction for the aroma molecule.

6. The computer-implemented method of claim 5, wherein the odorant descriptors comprise physicochemical descriptors, topological descriptors, EVA descriptors, Morgan fingerprint descriptors, or a combination thereof.

7. The computer-implemented method of claim 1, wherein the human olfactory evaluation is performed by a method comprising:
presenting to a subject, by a user interface, a set of odorant descriptions and a respective set of rating controls, and
receiving ratings entered by the subject using the rating controls, each rating being indicative of a descriptiveness of a respective odorant description for the aroma molecule, thereby
obtaining a set of odorant descriptions for the aroma molecule.

8. An apparatus for quality assessment of aroma molecules using single-molecule olfactory predictions, comprising:
an input unit configured to receive a data input, preferably a user interface, wherein the data input comprises at least one set of odorant descriptions obtained by olfactory prediction of an aroma molecule and a second set of odorant descriptions obtained by human olfactory evaluation;
a processing unit, preferably a processing unit comprising at least one processor, configured, specifically by programming, to compare the received set of odorant descriptions obtained by olfactory prediction of an aroma molecule to the second set of odorant descriptions obtained by olfactory prediction of the same aroma molecule and determine, specifically to calculate, the quality of aroma molecule based on the discrepancy between the at least one set of odorant descriptions obtained by olfactory prediction of the aroma molecule and the second set of odorant descriptions obtained by human olfactory evaluation; and
an output unit configured to output the quality assessment results of the aroma molecule to the user and/or to a data interface.

9. A system for quality assessment of aroma molecules using single-molecule olfactory predictions, comprising:
an apparatus according to claim 8; and
a web server configured to interface with a user via a webpage served by the web server and/or via an application program;
wherein the system is configured to provide a graphical user interface (GUI) to a user by the webpage and/or the application program.

10. A computer program comprising instructions which cause an apparatus and/or a system to perform the method of claim 1.

11. A computer-readable storage medium comprising instructions which cause an apparatus and/or a system to perform the method of claim 1.
